(19) United States
(12) Patent Application Publication
(10) Pub. No.: US 2026/0256459 A1
(43) Pub. Date: Sep. 3, 2026

(54) ULTRASOUND DIAGNOSTIC APPARATUS AND CONTROL METHOD OF ULTRASOUND DIAGNOSTIC APPARATUS

(71) Applicant: FUJIFILM Corporation, Tokyo (JP)

(72) Inventor: Tetsurou EBATA, Kanagawa (JP)

(73) Assignee: FUJIFILM Corporation, Tokyo (JP)

(21) Appl. No.: 19/553,408

(22) Filed: Mar. 1, 2026

(30) Foreign Application Priority Data
Mar. 3, 2025 (JP) 2025-032676

(51) Int. Cl. A61B 8/00; A61B 8/08

(52) U.S. Cl. CPC A61B 8/54; A61B 8/0883; A61B 8/4254; A61B 8/483

(57) ABSTRACT

Provided are an ultrasound diagnostic apparatus and a control method of an ultrasound diagnostic apparatus, in which a user can easily acquire an ultrasound image representing a target cross section.

An ultrasound diagnostic apparatus includes a measurement position calculation unit that calculates a measurement position in real space based on a first position and a first posture of an ultrasound probe, in which a first ultrasound image is acquired, and a measurement position in the first ultrasound image; a probe position/posture calculation unit that calculates a second posture of the ultrasound probe in a case where a second ultrasound image is acquired from the first posture by using conversion information and that calculates a second position of the ultrasound probe in which a cross section drawn at the second posture passes through the measurement position in the real space; and a probe scanning guide unit that guides a user to scan the ultrasound probe based on the second position and the second posture of the ultrasound probe, and a current position and a current posture of the ultrasound probe.

### CROSS-REFERENCE TO RELATED APPLICATIONS

The present application claims priority under 35 U.S.C. § 119 to Japanese Patent Application No. 2025-032676, filed on Mar. 3, 2025. The above application is hereby expressly incorporated by reference, in its entirety, into the present application.

### BACKGROUND OF THE INVENTION

### 1. Field of the Invention

The present invention relates to an ultrasound diagnostic apparatus that images a heart of a subject and a control method of the ultrasound diagnostic apparatus.

### 2. Description of the Related Art

In the related art, a cardiac output is calculated by capturing an ultrasound image representing a tomographic plane of a heart of a subject using an ultrasound diagnostic apparatus and analyzing the captured ultrasound image. The cardiac output is usually calculated by performing calculation steps of (1) measuring a diameter of a left ventricular outflow tract in a first ultrasound image representing a so-called parasternal left ventricular long-axis tomographic plane at a mid-systolic phase of the heart, and calculating a cross-sectional area of the left ventricular outflow tract, (2) calculating a velocity-time integral value of a blood flow of the left ventricular outflow tract with respect to a so-called cardiac apex five-chamber tomographic plane or a so-called cardiac apex three-chamber tomographic plane by a so-called pulse Doppler method, (3) calculating a so-called stroke volume by multiplying the cross-sectional area of the left ventricular outflow tract by the velocity-time integral value of the blood flow of the left ventricular outflow tract, and (4) calculating the cardiac output by multiplying the stroke volume by a heart rate.

As described above, in a case of calculating the cardiac output, it is usually necessary to capture a first ultrasound image representing a first cross section and a second ultrasound image representing a second cross section. A position and a posture of the ultrasound probe for capturing each cross section vary depending on the subject, and a skill level is required to appropriately capture the cross section. Therefore, for example, as disclosed in JP2019-048211A, a technique of displaying a direction in which the ultrasound probe is to be moved to capture a target cross section in the ultrasound image has been developed. In JP2019-048211A, an ultrasound image of a target cross section is acquired in advance, and a direction in which the ultrasound probe is to be moved is calculated based on a position and a posture of the ultrasound probe in a case of acquiring the ultrasound image.

### SUMMARY OF THE INVENTION

However, a user such as a doctor or a technician who is not skilled in the ultrasound examination may not be able to capture the target cross section in the first place, and even in a case where the technique of JP2019-048211A is used, may not be able to calculate the direction of the ultrasound probe for capturing the target cross section. In addition, even a user who is skilled in the ultrasound examination may need to perform scanning within a certain range to search for an appropriate position and posture of the ultrasound probe to capture the target cross section, which may require a large amount of examination time.

The present invention has been made to solve the above-described problem in the related art, and an object of the present invention is to provide an ultrasound diagnostic apparatus and a control method of the ultrasound diagnostic apparatus, in which a user can easily acquire an ultrasound image representing a target cross section.

According to the following configuration, the above object can be achieved.
[1] An ultrasound diagnostic apparatus that acquires a first ultrasound image and a second ultrasound image in which a first cross section and a second cross section of a heart of a subject, which are different from each other, are respectively imaged, the ultrasound diagnostic apparatus comprising:
an ultrasound probe;
a probe position/posture sensor that detects a position and a posture of the ultrasound probe;
a measurement position calculation unit that calculates a measurement position of a measurement target object in real space based on a first position and a first posture of the ultrasound probe, which are detected by the probe position/posture sensor in a case where the first ultrasound image is acquired, and a measurement position of the measurement target object in the first ultrasound image;
a probe position/posture calculation unit that calculates a second posture of the ultrasound probe in a case where the second ultrasound image is acquired from the first posture by using conversion information for converting the first cross section into the second cross section and that calculates a second position of the ultrasound probe in which a cross section drawn by the ultrasound probe at the second posture passes through the measurement position of the measurement target object in the real space; and
a probe scanning guide unit that guides a user to perform scanning with the ultrasound probe such that the ultrasound probe is at the second position and the second posture based on the second position and the second posture of the ultrasound probe, which are calculated by the probe position/posture calculation unit, and a current position and a current posture of the ultrasound probe, which are detected by the probe position/posture sensor.
[2] The ultrasound diagnostic apparatus according to [1],
in which one of the first cross section and the second cross section is a parasternal left ventricular long-axis tomographic plane, and the other is a cardiac apex five-chamber tomographic plane.
[3] The ultrasound diagnostic apparatus according to [1], further comprising:
a conversion information memory that stores predetermined conversion information,
in which the probe position/posture calculation unit calculates the second position by using the conversion information stored in the conversion information memory.
[4 ] The ultrasound diagnostic apparatus according to [1], further comprising:
a conversion information calculation unit that calculates the conversion information based on a plurality of ultrasound images in which the heart is imaged,
in which the probe position/posture calculation unit calculates the second position and the second posture by using the conversion information calculated by the conversion information calculation unit.
[5] The ultrasound diagnostic apparatus according to [4],
in which the conversion information calculation unit
generates three-dimensional data of the heart based on the plurality of ultrasound images, and
calculates the conversion information based on the generated three-dimensional data.
[6] The ultrasound diagnostic apparatus according to [4],
in which the conversion information calculation unit
calculates, by performing image analysis, an indicator indicating a second cross section likeness for the plurality of ultrasound images passing through the measurement position of the measurement target object in the real space calculated by the measurement position calculation unit, and
calculates the conversion information based on the ultrasound image having a highest calculated indicator and the first ultrasound image.
[7] The ultrasound diagnostic apparatus according to any one of [1] to [6], further comprising: a body movement determination unit that determines presence or absence of body movement of the subject,
in which the probe scanning guide unit stops the guiding of the scanning with the ultrasound probe in a case where the body movement determination unit determines that the body movement is present.
[8] The ultrasound diagnostic apparatus according to [7],
in which the body movement determination unit determines that the body movement is present in a case where a similarity between a past ultrasound image and a current ultrasound image, in which the position and the posture of the ultrasound probe detected by the probe position/posture sensor are the same, is equal to or less than a predetermined similarity threshold value.
[9] The ultrasound diagnostic apparatus according to [7],
in which the body movement determination unit
generates three-dimensional data of the heart based on a plurality of ultrasound images in which the heart is imaged, and
determines that the body movement is present in a case where a similarity between the current ultrasound image and a two-dimensional image obtained from the three-dimensional data based on a cross section corresponding to the current position and the current posture of the ultrasound probe detected by the probe position/posture sensor is equal to or less than a predetermined similarity threshold value.
[10] The ultrasound diagnostic apparatus according to [7], further comprising:
an optical camera that images the subject,
in which the body movement determination unit determines presence or absence of the body movement of the subject based on an optical image acquired by the optical camera.
[11] The ultrasound diagnostic apparatus according to any one of [1] to [6], further comprising:
a body movement amount calculation unit that calculates a body movement amount of the subject; and
a probe position/posture correction unit that corrects the second position and the second posture of the ultrasound probe calculated by the probe position/posture calculation unit based on the body movement amount calculated by the body movement amount calculation unit,
in which the probe scanning guide unit guides the scanning with the ultrasound probe based on the second position and the second posture of the ultrasound probe corrected by the probe position/posture correction unit and the current position and the current posture of the ultrasound probe detected by the probe position/posture sensor.
[12] The ultrasound diagnostic apparatus according to any one of [1] to [11], further comprising:
an optical camera that images the subject,
in which the probe scanning guide unit instructs the user on a scanning direction of the ultrasound probe with the subject as a reference, based on an optical image acquired by the optical camera.
[13] A control method of an ultrasound diagnostic apparatus that acquires a first ultrasound image and a second ultrasound image in which a first cross section and a second cross section of a heart of a subject, which are different from each other, are respectively imaged, the control method comprising:
detecting a position and a posture of an ultrasound probe;
calculating a measurement position of a measurement target object in real space based on a first position and a first posture of the ultrasound probe, which are detected in a case where the first ultrasound image is acquired, and a measurement position of the measurement target object in the first ultrasound image;
calculating a second posture of the ultrasound probe in a case where the second ultrasound image is acquired from the first posture by using conversion information for converting the first cross section into the second cross section;
calculating a second position of the ultrasound probe in which a cross section drawn by the ultrasound probe at the second posture passes through the measurement position of the measurement target object in the real space; and
guiding a user to perform scanning with the ultrasound probe such that the ultrasound probe is at the second position and the second posture based on the second position and the second posture of the ultrasound probe, which are calculated, and a current position and a current posture of the ultrasound probe, which are detected.

In the present invention, the ultrasound diagnostic apparatus comprises an ultrasound probe; a probe position/posture sensor that detects a position and a posture of the ultrasound probe; a measurement position calculation unit that calculates a measurement position of a measurement target object in real space based on a first position and a first posture of the ultrasound probe, which are detected by the probe position/posture sensor in a case where the first ultrasound image is acquired, and a measurement position of the measurement target object in the first ultrasound image; a probe position/posture calculation unit that calculates a second posture of the ultrasound probe in a case where the second ultrasound image is acquired from the first posture by using conversion information for converting the first cross section into the second cross section and that calculates a second position of the ultrasound probe in which a cross section drawn by the ultrasound probe at the second posture passes through the measurement position of the measurement target object in the real space; and a probe scanning guide unit that guides a user to perform scanning with the ultrasound probe such that the ultrasound probe is at the second position and the second posture based on the second position and the second posture of the ultrasound probe, which are calculated by the probe position/posture calculation unit, and a current position and a current posture of the ultrasound probe, which are detected by the probe position/posture sensor. Therefore, the user can easily acquire an ultrasound image representing a target cross section.

### BRIEF DESCRIPTION OF THE DRAWINGS

FIG. 1 is a block diagram showing a configuration of an ultrasound diagnostic apparatus according to Embodiment 1 of the present invention.

FIG. 2 is a block diagram showing an internal configuration of a transmission/reception circuit in Embodiment 1 of the present invention.

FIG. 3 is a block diagram showing an internal configuration of an image generation unit in Embodiment 1 of the present invention.

FIG. 4 is a diagram showing an example of an ultrasound image representing a parasternal left ventricular long-axis tomographic plane.

FIG. 5 is a diagram showing an example of an ultrasound image representing a cardiac apex five-chamber tomographic plane.

FIG. 6 is a diagram showing an example of a measurement position in the ultrasound image representing the parasternal left ventricular long-axis tomographic plane.

FIG. 7 is a diagram showing an example of a measurement position in the ultrasound image representing the cardiac apex five-chamber tomographic plane.

FIG. 8 is a diagram schematically showing an example of a second cross section obtained by converting the first cross section.

FIG. 9 is a diagram showing a display example of a message for guiding scanning with the ultrasound probe.

FIG. 10 is a flowchart showing an operation of the ultrasound diagnostic apparatus according to Embodiment 1 of the present invention.

FIG. 11 is a block diagram showing a configuration of an ultrasound diagnostic apparatus according to Embodiment 2 of the present invention.

FIG. 12 is a block diagram showing a configuration of an ultrasound diagnostic apparatus according to Embodiment 3 of the present invention.

FIG. 13 is a block diagram showing a configuration of an ultrasound diagnostic apparatus according to Embodiment 4 of the present invention.

FIG. 14 is a block diagram showing a configuration of an ultrasound diagnostic apparatus according to Embodiment 5 of the present invention.

### DESCRIPTION OF THE PREFERRED EMBODIMENTS

Hereinafter, embodiments of the present invention will be described based on the accompanying drawings.

The description of configuration requirements described below is given on the basis of a representative embodiment of the present invention, but the present invention is not limited to such an embodiment.

Note that, in the present specification, a numerical range represented by using “to” means a range including numerical values before and after “to” as a lower limit value and an upper limit value.

In the present specification, “the same” includes an error range generally allowed in the technical field.

### Embodiment 1

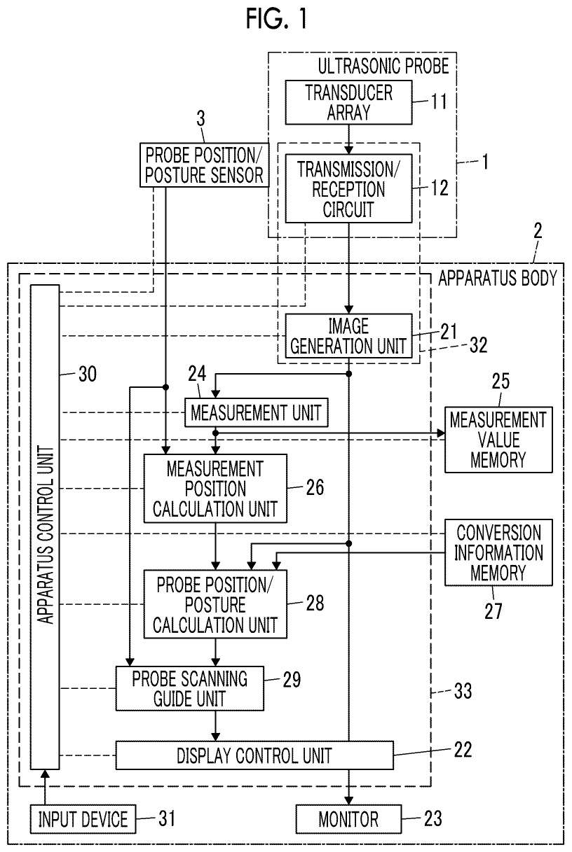

FIG. 1 shows a configuration of an ultrasound diagnostic apparatus according to Embodiment 1 of the present invention. The ultrasound diagnostic apparatus comprises an ultrasound probe 1 and an apparatus body 2 that are connected to each other by so-called wired communication or so-called wireless communication. In addition, the ultrasound diagnostic apparatus comprises a probe position/posture sensor 3 attached to the ultrasound probe 1.

The ultrasound probe 1 comprises a transducer array 11 and a transmission/reception circuit 12 connected to the transducer array 11.

The apparatus body 2 comprises an image generation unit 21 connected to the transmission/reception circuit 12. In the apparatus body 2, a display control unit 22 and a monitor 23 are sequentially connected to the image generation unit 21. A measurement unit 24 is connected to the image generation unit 21. A measurement value memory 25 is connected to the measurement unit 24. A measurement position calculation unit 26 is connected to the probe position/posture sensor 3 and the measurement unit 24. In addition, the apparatus body 2 comprises a conversion information memory 27. A probe position/posture calculation unit 28 is connected to the image generation unit 21, the measurement position calculation unit 26, and the conversion information memory 27. A probe scanning guide unit 29 is connected to the probe position/posture sensor 3 and the probe position/posture calculation unit 28. The probe scanning guide unit 29 is connected to the display control unit 22. In addition, an apparatus control unit 30 is connected to the probe position/posture sensor 3, the transmission/reception circuit 12, the image generation unit 21, the display control unit 22, the measurement unit 24, the measurement value memory 25, the measurement position calculation unit 26, the conversion information memory 27, the probe position/posture calculation unit 28, and the probe scanning guide unit 29. An input device 31 is connected to the apparatus control unit 30.

The transmission/reception circuit 12 and the image generation unit 21 constitute an image acquisition unit 32. In addition, a processor 33 for the apparatus body 2 is configured by the image generation unit 21, the display control unit 22, the measurement unit 24, the measurement position calculation unit 26, the probe position/posture calculation unit 28, the probe scanning guide unit 29, and the apparatus control unit 30.

The transducer array 11 of the ultrasound probe 1 has a plurality of ultrasonic transducers arranged in a one-dimensional or two-dimensional manner. According to a drive signal supplied from the transmission/reception circuit 12, each of the ultrasonic transducers transmits an ultrasonic wave and receives an ultrasound echo from the subject to output a signal based on the ultrasound echo. Each ultrasound transducer is configured by, for example, forming electrodes at both ends of a piezoelectric material consisting of piezoelectric ceramic represented by lead zirconate titanate (PZT), a polymer piezoelectric element represented by poly vinylidene di fluoride (PVDF), piezoelectric single crystal represented by lead magnesium niobate-lead titanate (PMN-PT), and the like.

The image acquisition unit 32, which is composed of the transmission/reception circuit 12 and the image generation unit 21, acquires ultrasound images of a plurality of frames as a moving image in which a heart of the subject is imaged, by transmitting and receiving ultrasound beams using the ultrasound probe 1.

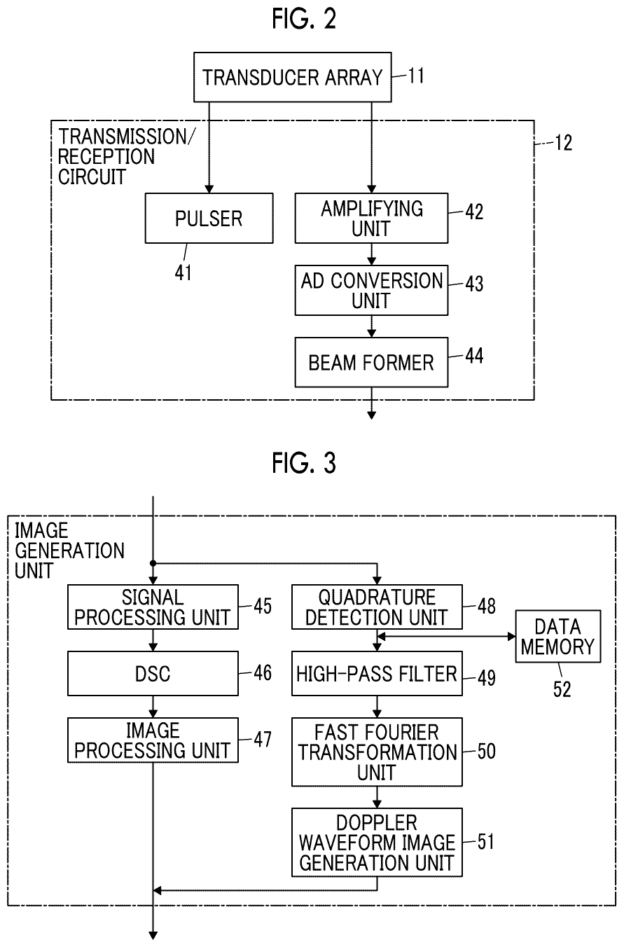

The transmission/reception circuit 12 transmits the ultrasound waves from the transducer array 11 and generates a sound ray signal based on reception signals acquired by the transducer array 11 under control of the apparatus control unit 30. As shown in FIG. 2, the transmission/reception circuit 12 includes a pulser 41 connected to the transducer array 11, and an amplifying unit 42, an analog-to-digital (AD) conversion unit 43, and a beam former 44 that are sequentially connected in series to the transducer array 11.

The pulser 41 includes, for example, a plurality of pulse generators, adjusts a delay amount of each drive signal based on a transmission delay pattern selected in accordance with a control signal from the apparatus control unit 30 so that the ultrasound waves transmitted from the plurality of ultrasound transducers of the transducer array 11 form an ultrasound beam, and supplies each drive signal to the plurality of ultrasound transducers. As described above, in a case in which a pulsed or continuous wave-like voltage is applied to the electrodes of the ultrasound transducer of the transducer array 11, the piezoelectric material expands and contracts to generate pulsed or continuous wave-like ultrasound from each of the ultrasound transducers, whereby the ultrasound beam is formed from the combined wave of the ultrasound.

The transmitted ultrasound beam is, for example, reflected by a target such as a part of the subject and propagates toward the transducer array 11 of the ultrasound probe 1. The ultrasound echo propagating toward the transducer array 11 in this way is received by each of the ultrasound transducers constituting the transducer array 11. In such a case, each of the ultrasound transducers constituting the transducer array 11 receives the propagating ultrasound echo to expand and contract, generates the reception signal, which is an electrical signal, and outputs these reception signals to the amplifying unit 42.

The amplifying unit 42 amplifies the signal input from each of the ultrasound transducers constituting the transducer array 11 and transmits the amplified signal to the AD conversion unit 43. The AD conversion unit 43 converts the signal transmitted from the amplifying unit 42 into digital reception data. The beam former 44 performs so-called reception focus processing by applying and adding the delay to each reception data received from the AD conversion unit 43. By the reception focus processing, each reception data, which is converted by the AD conversion unit 43, is phase-added, and the sound ray signal in which the focus of the ultrasound echo is narrowed down is acquired.

As shown in FIG. 3, the image generation unit 21 has a configuration in which a signal processing unit 45, a digital scan converter (DSC) 46, and an image processing unit 47 are connected in series, and a configuration in which a quadrature detection unit 48, a high-pass filter 49, a fast Fourier transformation unit 50, and a Doppler waveform image generation unit 51 are connected in series, and the configuration including the signal processing unit 45 to the image processing unit 47 and the configuration including the quadrature detection unit 48 to the Doppler waveform image generation unit 51 are connected in parallel to each other. In addition, a data memory 52 is connected to the quadrature detection unit 48.

The signal processing unit 45 corrects attenuation by distance of the sound ray signal received from the transmission/reception circuit 12 in accordance with depths of reflection positions of the ultrasound waves using a sound speed value set by the apparatus control unit 30 and then performs envelope detection processing on the sound ray signal to generate a B-mode image signal that is tomographic image information related to tissues inside the subject.

The DSC 46 converts (raster-converts) the B-mode image signal, which is generated by the signal processing unit 45, into the image signal in accordance with a normal television signal scanning method.

The image processing unit 47 performs various necessary image processing such as gradation processing on the B-mode image signal input from the DSC 46, and then transmits the B-mode image signal to the display control unit 22, the measurement unit 24, and the probe position/posture calculation unit 28. Hereinafter, the B-mode image signal, which is image-processed by the image processing unit 47, will be referred to as an ultrasound image.

The quadrature detection unit 48 performs orthogonal detection on the sound ray signal by mixing the sound ray signal received from the transmission/reception circuit 12 with a carrier signal of a reference frequency, and converts the sound ray signal into a complex signal.

The high-pass filter 49 functions as a so-called wall filter, and removes a frequency component derived from the motion of the body tissue inside the subject, from the complex signal generated by the quadrature detection unit 48.

The fast Fourier transformation unit 50 performs a Fourier transform on the complex signal of a plurality of sample points to perform frequency analysis, obtains the blood flow velocity, and generates a spectrum signal.

The Doppler waveform image generation unit 51 generates a Doppler waveform image signal by aligning the spectrum signals generated by the fast Fourier transformation unit 50 on a time axis and expressing the magnitude of each frequency component in brightness. In the Doppler waveform image, the time axis is shown on the horizontal axis, the Doppler shift frequency, that is, the flow velocity is shown on the vertical axis, and the brightness of the waveform represents the power at each frequency component.

The data memory 52 stores the complex signal converted from the reception data by the quadrature detection unit 48. In addition, as the data memory 52, for example, a recording medium such as a flash memory, a hard disk drive (HDD), a solid-state drive (SSD), a flexible disk (FD), a magneto-optical disk (MO disk), a magnetic tape (MT), a random-access memory (RAM), a compact disc (CD), a digital versatile disc (DVD), a secure digital card (SD card), or a universal serial bus memory (USB memory) can be used.

The processing of generating the Doppler waveform image by the quadrature detection unit 48, the high-pass filter 49, the fast Fourier transformation unit 50, and the Doppler waveform image generation unit 51 is performed by transmitting the ultrasound waves in a pulse shape to the transducer array 11 by sending the drive signal under the control of the transmission/reception circuit 12 and the apparatus control unit 30, and the Doppler waveform image is generated in a so-called Doppler gate set on the ultrasound image by the input from the user via the input device 31 or the like.

Meanwhile, a technique of calculating a cardiac output by capturing an ultrasound image U representing a tomographic plane of a heart of a subject using an ultrasound diagnostic apparatus and analyzing the captured ultrasound image U is known. The cardiac output is usually calculated by performing calculation steps of (1) measuring a diameter of a left ventricular outflow tract in an ultrasound image U representing a so-called parasternal left ventricular long-axis tomographic plane at a mid-systolic phase of the heart, and calculating a cross-sectional area of the left ventricular outflow tract, (2) calculating a velocity-time integral value of a blood flow of the left ventricular outflow tract with respect to a so-called cardiac apex five-chamber tomographic plane or a so-called cardiac apex three-chamber tomographic plane by a so-called pulse Doppler method, (3) calculating a so-called stroke volume by multiplying the cross-sectional area of the left ventricular outflow tract by the velocity-time integral value of the blood flow of the left ventricular outflow tract, and (4) calculating the cardiac output by multiplying the stroke volume by a heart rate.

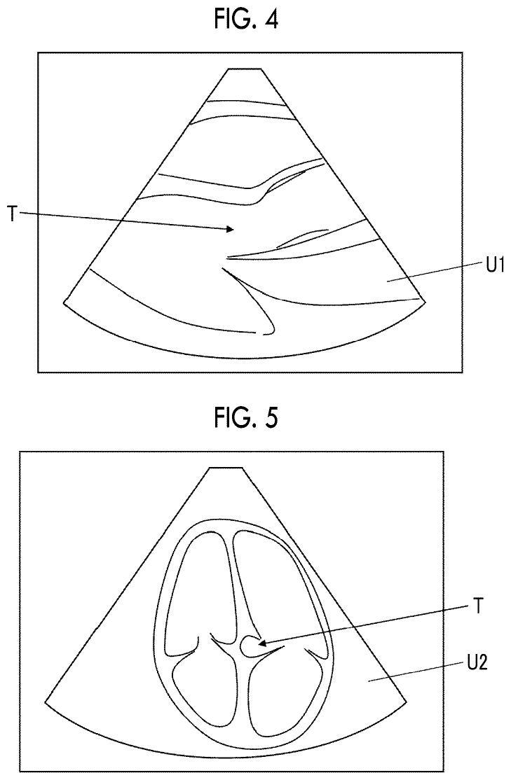

In the present invention, the image acquisition unit 32 acquires a first ultrasound image and a second ultrasound image in which a first cross section and a second cross section of a heart of a subject, which are different from each other, are imaged, respectively, for calculating the cardiac output. The image acquisition unit 32 can acquire, as the first ultrasound image representing the first cross section or the second ultrasound image representing the second cross section, for example, an ultrasound image U1 representing a so-called parasternal left ventricular long-axis tomographic plane that is longitudinal to a so-called left ventricular outflow tract T of the heart as shown in FIG. 4, and an ultrasound image U2 representing a so-called cardiac apex five-chamber tomographic plane that is transverse to the so-called left ventricular outflow tract T as shown in FIG. 5. In addition, the image acquisition unit 32 can acquire, for example, an ultrasound image representing a so-called cardiac apex three-chamber tomographic plane that is longitudinal to the left ventricular outflow tract T as the first ultrasound image or the second ultrasound image, although not shown.

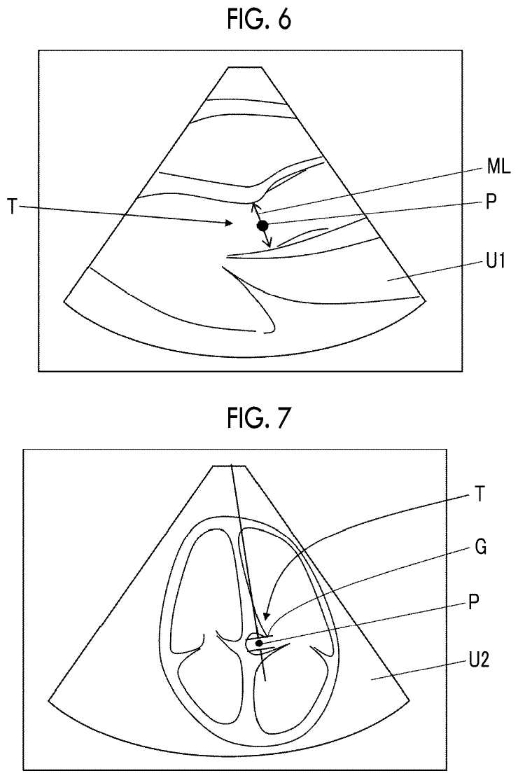

The measurement unit 24 performs the measurement on the heart of the subject by using the ultrasound image generated by the image generation unit 21. In a case where the ultrasound image U1 representing the parasternal left ventricular long-axis tomographic plane shown in FIG. 4 is generated by the image generation unit 21, in the measurement unit 24, for example, as shown in FIG. 6, a measurement line ML is disposed on the left ventricular outflow tract T shown in the ultrasound image U1, and a length of the disposed measurement line ML is measured as a diameter of the left ventricular outflow tract T. In a case where the ultrasound image U2 representing the cardiac apex five-chamber tomographic plane shown in FIG. 5 is generated by the image generation unit 21, the measurement unit 24 can calculate, for example, as shown in FIG. 7, the velocity-time integral value of the blood flow of the left ventricular outflow tract T based on the Doppler waveform image generated by the image generation unit 21 in a Doppler gate G disposed on the left ventricular outflow tract T shown in the ultrasound image U2. In addition, in a case where an ultrasound image representing a cardiac apex three-chamber tomographic plane (not shown) is generated instead of the ultrasound image U2 representing the cardiac apex five-chamber tomographic plane, the Doppler gate G can be disposed on the left ventricular outflow tract T shown in the ultrasound image, and the velocity-time integral value of the blood flow of the left ventricular outflow tract T in the Doppler gate G can be calculated in the same manner as in a case where the ultrasound image U2 representing the cardiac apex five-chamber tomographic plane is generated.

The measurement value memory 25 is a memory that stores the measurement value acquired by the measurement unit 24. The user can measure the cardiac output of the subject by using the measurement value stored in the measurement value memory 25. As the measurement value memory 25, for example, recording media such as a flash memory, an HDD, an SSD, an FD, an MO disk, an MT, a RAM, a CD, a DVD, an SD card, or a USB memory can be used.

The probe position/posture sensor 3 detects the position and the posture of the ultrasound probe 1. The probe position/posture sensor 3 can detect the position of the ultrasound probe 1 with respect to any point outside the subject or any point on the subject, for example, in the form of three-dimensional position coordinates. In addition, the probe position/posture sensor 3 can detect the inclination angle of the ultrasound probe 1 in a form of three-dimensional angle coordinates with respect to, for example, a vertical downward direction as the posture of the ultrasound probe 1.

The probe position/posture sensor 3 can include, for example, a known sensor device that detects the position and the posture of an object, such as a so-called acceleration sensor, a gyro sensor, a magnetic sensor, and a global positioning system (GPS). Depending on the type of the probe position/posture sensor 3, the probe position/posture sensor 3 can be installed away from the ultrasound probe 1 instead of being attached to the ultrasound probe 1. In addition, the probe position/posture sensor 3 can include, for example, an optical camera that acquires an optical image in which the ultrasound probe 1 is shown, and an optical image analysis unit that analyzes the acquired optical image to detect the position and the posture of the ultrasound probe 1. In this case, for example, a figure used as a so-called augmented reality (AR) marker such as a so-called ArUco (Augmented Reality University of Cordoba) can be disposed on a housing of the ultrasound probe 1, and the optical image analysis unit can analyze the figure to detect the position and the posture of the ultrasound probe 1.

The measurement position calculation unit 26 calculates the measurement position of the measurement target object in the real space based on the first position and the first posture of the ultrasound probe 1 detected by the probe position/posture sensor 3 in a case of acquiring the first ultrasound image and the measurement position of the measurement target object in the first ultrasound image.

In a case where the ultrasound image U1 representing the parasternal left ventricular long-axis tomographic plane is generated as the first ultrasound image and the diameter of the left ventricular outflow tract T is measured by the measurement unit 24, in the measurement position calculation unit 26, for example, as shown in FIG. 6, a midpoint of the measurement line ML disposed on the left ventricular outflow tract T can be set as the measurement position P of the measurement target object in the first ultrasound image. In addition, in a case where the ultrasound image U2 representing the cardiac apex five-chamber tomographic plane is generated as the first ultrasound image and the velocity-time integral value of the blood flow of the left ventricular outflow tract T is measured by the measurement unit 24, the measurement position calculation unit 26 can set, for example, as shown in FIG. 7, a center position of the Doppler gate G disposed on the left ventricular outflow tract T as the measurement position P of the measurement target object in the first ultrasound image.

The measurement position calculation unit 26 can calculate the measurement position P of the measurement target object in the real space, that is, three-dimensional position coordinates representing the measurement position P of the measurement target object in the three-dimensional space based on, for example, two-dimensional coordinates of the measurement position P set in this way in the first ultrasound image and three-dimensional position coordinates representing the first position of the ultrasound probe 1 and three-dimensional angle coordinates representing the first posture of the ultrasound probe 1 detected by the probe position/posture sensor 3.

The conversion information memory 27 is a memory that stores conversion information for converting the first cross section represented by the first ultrasound image into the second cross section represented by the second ultrasound image in advance. The conversion information memory 27 can store the conversion information in a form of, for example, a so-called rotation matrix, a so-called quaternion, or a so-called Euler angle. As the conversion information memory 27, for example, recording media such as a flash memory, an HDD, an SSD, an FD, an MO disk, an MT, a RAM, a CD, a DVD, an SD card, or a USB memory can be used.

The probe position/posture calculation unit 28 calculates the second posture of the ultrasound probe 1 in a case where the second ultrasound image is acquired from the first posture by using the conversion information stored in the conversion information memory 27 for converting the first cross section into the second cross section, and calculates the second position of the ultrasound probe 1 in which the cross section drawn by the ultrasound probe 1 at the second posture passes through the measurement position P of the measurement target object in the real space.

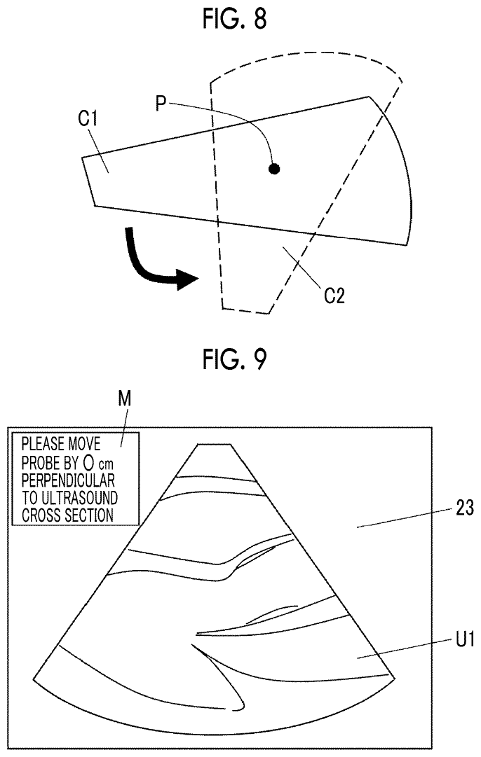

More specifically, the probe position/posture calculation unit 28 can calculate the second cross section C2 by, for example, as schematically shown in FIG. 8, applying the conversion information such as the rotation matrix to the first cross section C1 to rotate the first cross section C1 in the real space, and further moving the cross section after the rotation in the real space to pass through the measurement position P calculated by the measurement position calculation unit 26. In this case, the probe position/posture calculation unit 28 can move the cross section rotated by the conversion information such that the measurement position P is located at a center portion of the second cross section C2, for example.

The probe position/posture calculation unit 28 calculates the second position and the second posture of the ultrasound probe 1 for acquiring the second ultrasound image representing the second cross section C2 based on the second cross section C2 calculated in this way. The probe position/posture calculation unit 28 can specify, for example, a center line that extends from a shallowest portion to a deepest portion of the second cross section C2 and passes through the center of the second cross section C2, calculate a point on the center line corresponding to the shallowest portion of the second cross section C2 as the second position, and calculate a direction in which the center line extends from the shallowest portion to the deepest portion of the second cross section C2 as the second posture.

The probe scanning guide unit 29 guides the user to perform scanning with the ultrasound probe 1 such that the ultrasound probe 1 is at the second position and the second posture based on the second position and the second posture of the ultrasound probe 1 calculated by the probe position/posture calculation unit 28 and the current position and the current posture of the ultrasound probe 1, which are detected by the probe position/posture sensor 3. The probe scanning guide unit 29 can guide the scanning with the ultrasound probe 1 by, for example, as shown in FIG. 9, displaying a message M such as “please move the probe by ○ cm perpendicular to the ultrasound cross section” on the monitor 23. In addition, in a case where the ultrasound diagnostic apparatus comprises a speaker (not shown), the probe scanning guide unit 29 can also guide the scanning with the ultrasound probe 1 by a voice via the speaker. In addition, in a case where the ultrasound probe 1 comprises a vibration device (not shown) such as a so-called vibration motor, the scanning with the ultrasound probe 1 can also be guided by the vibration of the ultrasound probe 1 caused by the vibration device.

The display control unit 22 performs predetermined processing on the first ultrasound image and the second ultrasound image acquired by the image acquisition unit 32, the message M for the probe scanning guide unit 29 to guide the scanning with the ultrasound probe 1, and the like under the control of the apparatus control unit 30, and displays the processed images on the monitor 23.

The monitor 23 displays the ultrasound image U or the like under the control of the display control unit 22 and includes, for example, a display device such as a liquid crystal display (LCD) or an organic electroluminescence display (organic EL display).

The input device 31 is an input device for the user to perform an input operation, and is configured by, for example, a device such as a keyboard, a mouse, a trackball, a touchpad, and a touch sensor disposed in a state of being superimposed on the monitor 23.

In the present embodiment, each processing in the processor 33 is executed by any computer. In addition, any computer may execute these types of processing by a processor as hardware, a program as software, or a combination thereof. In such a case, the processor is configured to execute various types of processing in the present embodiment in cooperation with the program, and may function as each unit or each means in the present embodiment. In addition, the execution order of the processing by the processor is not limited to the above-described order and may be changed as appropriate. Any computer may be a general-purpose computer, a computer for specific use, a workstation, or another system capable of executing each processing.

The processor 33 may be configured using one or more pieces of hardware, and the type of hardware is not limited. For example, the processor 33 may be composed of hardware such as a central processing unit (CPU), a micro processing unit (MPU), a programmable logic device such as a field programmable gate array (FPGA), a dedicated circuit for executing specific processing, such as an application specific integrated circuit (ASIC), a graphic processing unit (GPU), or a neural processing unit (NPU). The types of hardware may be a combination of different types of hardware. In a case in which the plurality of types of hardware are configured to execute one or a plurality of types of processing of a certain processor, the plurality of types of hardware may exist in devices physically separated from each other or may exist in the same device. Furthermore, in any of the embodiments, the order of each processing performed by the processor 33 is not limited to the above-described order, and may be changed as appropriate. The hardware is composed of an electric circuit (circuitry) in which circuit elements such as semiconductor elements are combined.

Further, the program may be software, such as firmware or a microcode. Furthermore, the program may be, for example, a program module group, and each function thereof may be implemented by a processor configured to execute each function. The program may be a program code or a plurality of code segments stored in one or a plurality of non-transitory computer-readable media (for example, a storage medium and other storages). The program may be stored in the plurality of non-transitory computer-readable media existing in physically separated devices. The program code or the code segment may represent any combination of procedures, functions, subprograms, routines, subroutines, modules, software packages, classes, instructions, data structures, or program statements. The program code or the code segments may be connected to other code segments or hardware circuits by transmitting and receiving information, data, an argument, a parameter, or content of a memory.

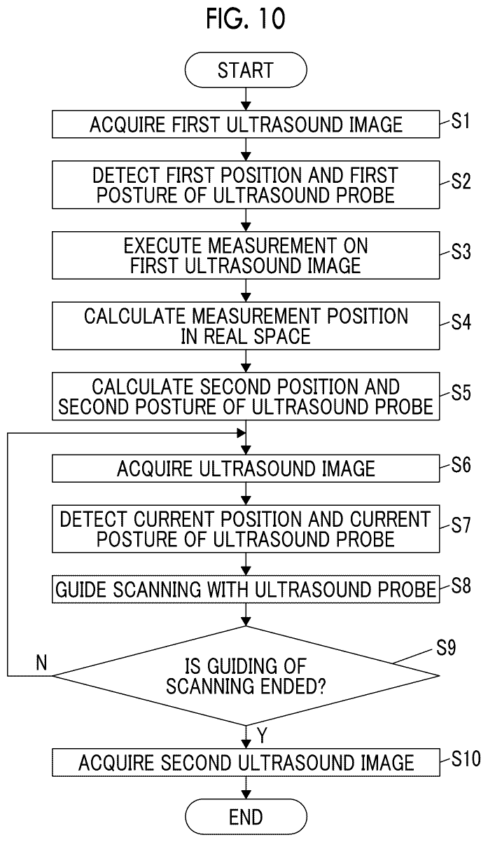

Hereinafter, an operation of the ultrasound diagnostic apparatus according to Embodiment 1 will be described with reference to a flowchart shown in FIG. 10.

In step S1, the image acquisition unit 32 acquires the first ultrasound image among the first ultrasound image representing the first cross section C1 and the second ultrasound image representing the second cross section C2 that are imaged in a case where the cardiac output of the subject is measured. In such a case, under the control of the apparatus control unit 30, the transmission and reception of the ultrasound from the plurality of transducers of the transducer array 11 are started in accordance with the drive signal from the pulser 41 of the transmission/reception circuit 12 of the ultrasound probe 1, the ultrasound echo from the subject is received by the plurality of transducers of the transducer array 11, and the reception signal as the analog signal is output to the amplifying unit 42, is amplified, and then is subjected to the AD conversion via the AD conversion unit 43 to acquire the reception data.

The reception focus processing is performed on the reception data by the beam former 44, the sound ray signal generated by the reception focusing processing is transmitted to the image generation unit 21 of the apparatus body 2, and thus the first ultrasound image is generated by the image generation unit 21. In this case, the signal processing unit 45 of the image generation unit 21 performs the correction of the attenuation in accordance with the depth of the reflection position of the ultrasound and the envelope detection processing on the sound ray signal, the DSC 46 performs the conversion into the image signal in accordance with the normal television signal scanning method, and the image processing unit 47 performs various types of necessary image processing, such as gradation processing. The first ultrasound image generated in step S1 in this way is transmitted to the display control unit 22, the measurement unit 24, and the probe position/posture calculation unit 28.

In step S2, the probe position/posture sensor 3 detects the first position and the first posture of the ultrasound probe 1 in the real space in a case of acquiring the first ultrasound image in step S1. The probe position/posture sensor 3 can detect, for example, three-dimensional position coordinates representing the first position of the ultrasound probe 1 in the real space and three-dimensional angle coordinates representing the first posture of the ultrasound probe 1 in the real space.

In step S3, the measurement unit 24 executes the measurement related to the heart of the subject on the respect to the first ultrasound image acquired in step S1. In a case where the ultrasound image U1 representing the parasternal left ventricular long-axis tomographic plane shown in FIG. 6 is acquired in step S1, in the measurement unit 24, for example, the measurement line ML is disposed on the left ventricular outflow tract T and a length of the disposed measurement line ML can be measured as a diameter of the left ventricular outflow tract T.

In addition, in a case where the ultrasound image U2 representing the cardiac apex five-chamber tomographic plane shown in FIG. 7 is acquired in step S1, the measurement unit 24 can calculate, for example, the velocity-time integral value of the blood flow of the left ventricular outflow tract T based on the Doppler waveform image acquired by the image acquisition unit 32 in the Doppler gate G disposed on the left ventricular outflow tract T.

In step S4, the measurement position calculation unit 26 calculates the measurement position P of the measurement target object in the real space based on the first position and the first posture of the ultrasound probe 1 detected in step S2 and the measurement position P of the measurement target object measured in step S3 in the first ultrasound image.

In a case where the ultrasound image U1 representing the parasternal left ventricular long-axis tomographic plane is generated as the first ultrasound image in step S1 and the diameter of the left ventricular outflow tract T is measured in step S3, in the measurement position calculation unit 26, for example, as shown in FIG. 6, a midpoint of the measurement line ML disposed on the left ventricular outflow tract T can be set as the measurement position P of the measurement target object in the first ultrasound image. In addition, in a case where the ultrasound image U2 representing the cardiac apex five-chamber tomographic plane is generated as the first ultrasound image in step S1 and the velocity-time integral value of the blood flow of the left ventricular outflow tract T is measured in step S3, the measurement position calculation unit 26 can set, for example, as shown in FIG. 7, a center position of the Doppler gate G disposed on the left ventricular outflow tract T as the measurement position P of the measurement target object in the first ultrasound image.

The measurement position calculation unit 26 can calculate the three-dimensional position coordinates representing the measurement position P of the measurement target object in the three-dimensional space as the measurement position P of the measurement target object in the real space based on, for example, two-dimensional coordinates of the measurement position P set in this way in the first ultrasound image and three-dimensional position coordinates representing the first position of the ultrasound probe 1 and three-dimensional angle coordinates representing the first posture of the ultrasound probe 1 detected in step S2.

In step S5, as schematically shown in FIG. 8, the probe position/posture calculation unit 28 calculates the second posture of the ultrasound probe 1 in a case where the second ultrasound image is acquired from the first posture by using the conversion information stored in the conversion information memory 27, and calculates the second position of the ultrasound probe 1 in which the cross section drawn by the ultrasound probe 1 at the second posture passes through the measurement position P of the measurement target object in the real space calculated in step S4.

More specifically, the probe position/posture calculation unit 28 can calculate the second cross section C2 by, for example, applying the conversion information such as the rotation matrix to the first cross section C1 to rotate the first cross section C1 in the real space, and further moving the cross section after the rotation in the real space to pass through the measurement position P in the real space calculated in step S4. In this case, the probe position/posture calculation unit 28 can move the cross section rotated by the conversion information such that the measurement position P is located at a center portion of the second cross section C2, for example. The probe position/posture calculation unit 28 calculates the second position and the second posture of the ultrasound probe 1 for acquiring the second ultrasound image representing the second cross section C2 based on the second cross section C2 calculated in this way.

As described above, since the second position and the second posture of the ultrasound probe 1 for acquiring the second ultrasound image representing the second cross section C2 are automatically calculated based on the first cross section C1, the user can easily obtain the second position and the second posture of the ultrasound probe 1 by performing only the work of capturing the first ultrasound image representing the first cross section C1.

In step S6, the image acquisition unit 32 acquires the ultrasound image representing the tomographic plane of the heart of the subject in the same manner as in step S1. The ultrasound image acquired in step S6 is transmitted to the display control unit 22.

In step S7, the probe position/posture sensor 3 detects the current position and the current posture of the ultrasound probe 1 in the real space in the same manner as in step S2. The current position and the current posture of the ultrasound probe 1 detected in step S7 are transmitted to the probe scanning guide unit 29.

In step S8, the probe scanning guide unit 29 guides the user to perform scanning with the ultrasound probe 1 such that the ultrasound probe 1 is at the second position and the second posture based on the second position and the second posture of the ultrasound probe 1 calculated in step S5 and the current position and the current posture of the ultrasound probe 1 detected in step S7. The probe scanning guide unit 29 can guide the scanning with the ultrasound probe 1 by, for example, as shown in FIG. 9, displaying a message M such as “please move the probe by ○ cm perpendicular to the ultrasound cross section” on the monitor 23.

In step S9, the apparatus control unit 30 determines whether or not to end the scanning with the ultrasound probe 1. The apparatus control unit 30 can determine to end the scanning with the ultrasound probe 1 in a case where the user inputs an instruction to end the scanning with the ultrasound probe 1 via the input device 31 by, for example, the user determining that the ultrasound probe 1 is at the second position and the second posture. The apparatus control unit 30 can determine to continue the scanning with the ultrasound probe 1 in a case where, for example, the instruction to end the scanning with the ultrasound probe 1 is not input from the user via the input device 31.

In a case where it is determined to continue the scanning with the ultrasound probe 1 in step S9, the process returns to step S6, and a new ultrasound image is acquired. Thereafter, in step S7, the current position and the current posture of the ultrasound probe 1 are detected, in step S8, the scanning with the ultrasound probe 1 is guided, and in step S9, it is determined whether or not to end the scanning with the ultrasound probe 1. In this way, processing of steps S6 to S9 is repeated as long as it is determined in step S9 to continue the scanning with the ultrasound probe 1. The user moves the ultrasound probe 1 on the body surface of the subject toward the second position and the second posture of the ultrasound probe 1 calculated in step S5 while checking the ultrasound image acquired in step S6 and displayed on the monitor 23, the message M displayed on the monitor 23 in step S8, and the like.

In a case where it is determined that the ultrasound probe 1 is at the second position and the second posture and the scanning with the ultrasound probe 1 is determined to be ended in step S9 by the input from the user via the input device 31 or the like, the process proceeds to step S10. In step S10, the image acquisition unit 32 acquires the second ultrasound image in the same manner as in step S1 and step S6.

The second ultrasound image is used for the measurement on the heart by the measurement unit 24. The measurement value obtained based on the second ultrasound image and the measurement value obtained in step S3 are used, for example, for calculating the cardiac output of the subject.

In a case where the processing of step S10 is completed in such a manner, the operation of the ultrasound diagnostic apparatus following the flowchart of FIG. 10 ends.

As described above, according to the ultrasound diagnostic apparatus according to Embodiment 1 of the present invention, since the measurement position calculation unit 26 calculates the measurement position P of the measurement target object in the real space based on the first position and the first posture of the ultrasound probe 1 detected by the probe position/posture sensor 3 in a case where the first ultrasound image is acquired and the measurement position P of the measurement target object in the first ultrasound image, the probe position/posture calculation unit 28 calculates the second posture of the ultrasound probe 1 in a case where the second ultrasound image is acquired from the first posture by using the conversion information for converting the first cross section C1 into the second cross section C2 and calculates the second position of the ultrasound probe 1 in which the cross section drawn by the ultrasound probe 1 at the second posture passes through the measurement position P of the measurement target object in the real space, and the probe scanning guide unit 29 guides the user to perform scanning with the ultrasound probe 1 such that the ultrasound probe 1 is at the second position and the second posture based on the second position and the second posture of the ultrasound probe 1 calculated by the probe position/posture calculation unit 28 and the current position and the current posture of the ultrasound probe 1, which are detected by the probe position/posture sensor 3, the second ultrasound image representing the second cross section C2 can be easily acquired.

A case has been described in which the transmission/reception circuit 12 is provided in the ultrasound probe 1, but the transmission/reception circuit 12 may be provided in the apparatus body 2.

Further, a case has been described in which the image generation unit 21 is provided in the apparatus body 2, but the image generation unit 21 may be provided in the ultrasound probe 1.

The apparatus body 2 may be a so-called stationary type, a portable type that is easily carried, or a so-called handheld type that is configured by, for example, a smartphone or a tablet type computer. In this way, the type of the device constituting the apparatus body 2 is not particularly limited.

Meanwhile, for example, the optimal measurement position P for measuring the diameter of the left ventricular outflow tract T in the ultrasound image U1 representing the parasternal left ventricular long-axis tomographic plane and the optimal measurement position P as a position for disposing the Doppler gate G in the ultrasound image U2 representing the cardiac apex five-chamber tomographic plane may be different from each other. Therefore, the probe position/posture calculation unit 28 can store, for example, a positional relationship between the measurement position P in the ultrasound image U1 representing the parasternal left ventricular long-axis tomographic plane and the measurement position P in the ultrasound image U2 representing the cardiac apex five-chamber tomographic plane in the real space as a correction value in advance, and correct the position of the second cross section C2 based on the correction value. The correction value can also be input by the user via the input device 31. The probe position/posture calculation unit 28 can also set the correction value based on the positional relationship between the measurement position P set in the ultrasound image U1 representing the parasternal left ventricular long-axis tomographic plane and the measurement position P set in the ultrasound image U2 representing the cardiac apex five-chamber tomographic plane in the past examination.

### Embodiment 2

Although an aspect in which the conversion information memory 27 stores the conversion information in advance has been described, the conversion information can also be calculated in the ultrasound diagnostic apparatus.

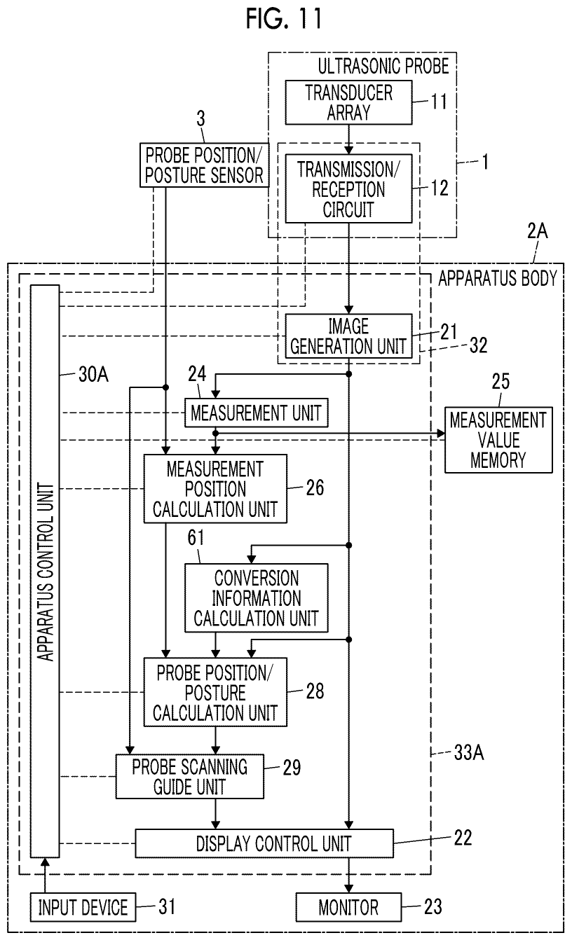

FIG. 11 shows a configuration of an ultrasound diagnostic apparatus of Embodiment 2. The ultrasound diagnostic apparatus according to Embodiment 2 comprises an apparatus body 2A instead of the apparatus body 2, as compared with the ultrasound diagnostic apparatus according to Embodiment 1 shown in FIG. 1. The apparatus body 2A according to Embodiment 2 is different from the apparatus body 2 according to Embodiment 1 in that the conversion information memory 27 is removed, a conversion information calculation unit 61 is further provided, and an apparatus control unit 30A is provided instead of the apparatus control unit 30.

In the apparatus body 2A, the conversion information calculation unit 61 is connected to the image generation unit 21. The conversion information calculation unit 61 is connected to the probe position/posture calculation unit 28 and the apparatus control unit 30A. A processor 33A for the apparatus body 2A is configured by the image generation unit 21, the display control unit 22, the measurement unit 24, the measurement position calculation unit 26, the probe position/posture calculation unit 28, the probe scanning guide unit 29, the apparatus control unit 30A, and the conversion information calculation unit 61.

The user moves the ultrasound probe 1 on the body surface of the subject after capturing the first ultrasound image to continuously capture a plurality of ultrasound images.

The conversion information calculation unit 61 calculates the conversion information based on a plurality of ultrasound images in which the heart of the subject is imaged. The conversion information calculation unit 61 can generate three-dimensional data of the heart of the subject based on the plurality of ultrasound images including the first ultrasound image acquired by the image acquisition unit 32, and calculate the conversion information based on the generated three-dimensional data.

More specifically, the conversion information calculation unit 61 stores, for example, a general three-dimensional model of the heart of the subject including the second cross section C2, and can specify the position and the inclination of the second cross section C2 in the three-dimensional data corresponding to the second cross section C2 in the three-dimensional model by matching the generated three-dimensional data with the stored three-dimensional model. The conversion information calculation unit 61 can calculate the conversion information based on the position and the inclination of the specified second cross section C2 and the position and the inclination of the first cross section C1 represented by the first ultrasound image acquired by the image acquisition unit 32.

Alternatively, the conversion information calculation unit 61 stores, for example, pattern data representing a general pattern of the second cross section C2, and can calculate a similarity between each of the plurality of cross sections of the generated three-dimensional data and the pattern data by matching the plurality of cross sections of the generated three-dimensional data with the stored pattern data, and specify a cross section having the highest similarity as the second cross section C2. It should be noted that the conversion information calculation unit 61 can also specify the cross section having the highest similarity by inputting the plurality of cross sections of the generated three-dimensional data to a so-called trained model in machine learning that has learned a relationship between the pattern data and a pattern of the plurality of cross sections in the heart. The conversion information calculation unit 61 can calculate the conversion information based on the position and the inclination of the specified second cross section C2 and the position and the inclination of the first cross section C1 represented by the first ultrasound image acquired by the image acquisition unit 32.

In addition, in a case where the user captures the plurality of ultrasound images while moving the ultrasound probe 1 at various positions and various postures on the body surface of the subject to capture the second ultrasound image representing the second cross section C2, the conversion information calculation unit 61 can calculate an indicator indicating a second cross section C2 likeness for the plurality of ultrasound images passing through the measurement position P of the measurement target object in the real space calculated by the measurement position calculation unit 26 by performing image analysis, specify the ultrasound image having the highest calculated indicator as the second ultrasound image, and calculate the conversion information based on the specified ultrasound image and the first ultrasound image.

In this case, the conversion information calculation unit 61 can calculate, for example, a similarity between the general pattern of the second cross section C2 and the plurality of ultrasound images as the indicator indicating the second cross section C2 likeness by a method using pattern matching or a trained model in machine learning.

The probe position/posture calculation unit 28 calculates the second position and the second posture by using the conversion information calculated by the conversion information calculation unit 61.

As described above, according to the ultrasound diagnostic apparatus according to Embodiment 2, since the conversion information calculation unit 61 calculates the conversion information based on the plurality of ultrasound images actually acquired, the conversion information according to the individual difference in the structure of the heart of the subject can be calculated.

### Embodiment 3

In general, the subject may move the body during the examination. In this case, the second position and the second posture of the ultrasound probe 1 for capturing the second ultrasound image change due to the body movement of the subject.

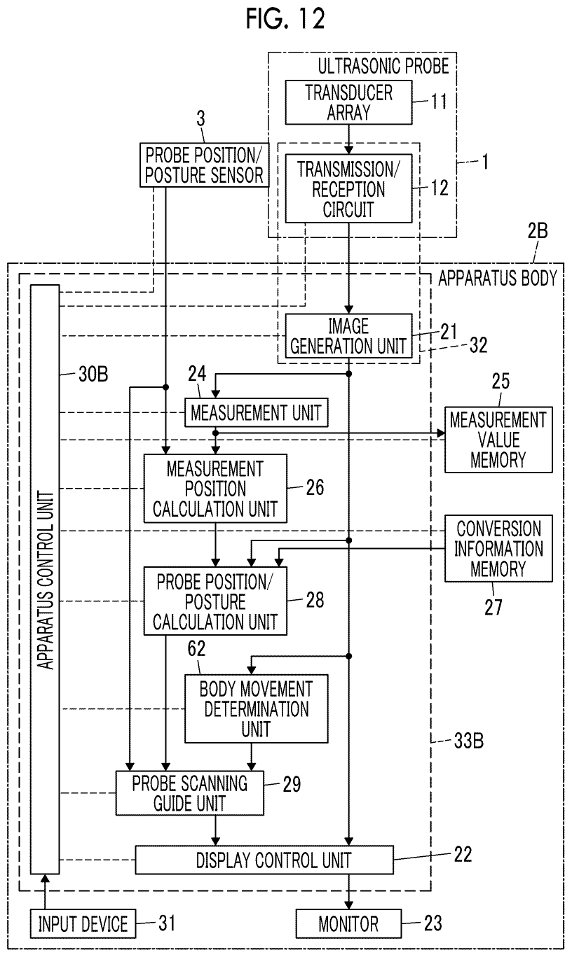

FIG. 12 illustrates a configuration of an ultrasound diagnostic apparatus of a third embodiment. The ultrasound diagnostic apparatus according to Embodiment 3 comprises an apparatus body 2B instead of the apparatus body 2, as compared to the ultrasound diagnostic apparatus according to Embodiment 1 shown in FIG. 1. The apparatus body 2B according to Embodiment 3 is different from the apparatus body 2 according to Embodiment 1 in that a body movement determination unit 62 is further provided, and an apparatus control unit 30B is provided instead of the apparatus control unit 30.

In the apparatus body 2B, the body movement determination unit 62 is connected to the image generation unit 21. The body movement determination unit 62 is connected to the probe scanning guide unit 29 and the apparatus control unit 30B. A processor 33B for the apparatus body 2B is configured by the image generation unit 21, the display control unit 22, the measurement unit 24, the measurement position calculation unit 26, the probe position/posture calculation unit 28, the probe scanning guide unit 29, the apparatus control unit 30B, and the body movement determination unit 62.

The body movement determination unit 62 determines presence or absence of the body movement of the subject based on the plurality of ultrasound images acquired by the image acquisition unit 32. The body movement determination unit 62 can determine that the body movement is present in a case where a similarity between a past ultrasound image in which the position and the posture of the ultrasound probe 1 detected by the probe position/posture sensor 3 are the same and a current ultrasound image is equal to or less than a similarity threshold value. In addition, the body movement determination unit 62 can determine that the body movement is not present in a case where the similarity is greater than the similarity threshold value.

Here, the past ultrasound image to be compared with the current ultrasound image refers to any one of the plurality of ultrasound images acquired from after the first ultrasound image is acquired to before the current ultrasound image is acquired. The body movement determination unit 62 can calculate the similarity by performing pattern matching processing on the past ultrasound image in which the position and the posture of the ultrasound probe 1 are the same and the current ultrasound image.

In addition, the body movement determination unit 62 can generate three-dimensional data of the heart based on the plurality of ultrasound images in which the heart of the subject is acquired by the image acquisition unit 32, and determine the presence or absence of the body movement of the subject based on the generated three-dimensional data. The body movement determination unit 62 can determine that the body movement is present in a case where a similarity between the current ultrasound image and a two-dimensional image obtained from the three-dimensional data based on a cross section corresponding to the current position and the current posture of the ultrasound probe 1 detected by the probe position/posture sensor 3 is equal to or less than a similarity threshold value. In this case, the body movement determination unit 62 can calculate the similarity by a method using pattern matching or a trained model in machine learning.

In a case where the body movement of the subject occurs while the user is moving the ultrasound probe 1 toward the second position and the second posture of the ultrasound probe 1 for acquiring the second ultrasound image, the second position and the second posture as the target may change. Therefore, the probe scanning guide unit 29 stops the guiding of the scanning with the ultrasound probe 1 in a case where the body movement determination unit 62 determines that the body movement is present. As a result, it is possible to prevent the ultrasound probe 1 from being scanned to the incorrect second position and the second posture due to the body movement of the subject.

In addition, in a case of stopping the guiding of the scanning with the ultrasound probe 1, the probe scanning guide unit 29 can notify the user that the guiding of the ultrasound probe 1 is stopped by, for example, displaying the message M on the monitor 23.

As described above, according to the ultrasound diagnostic apparatus according to Embodiment 3, since the body movement determination unit 62 determines presence or absence of the body movement of the subject and the probe scanning guide unit 29 stops the guiding of the scanning with the ultrasound probe 1 in a case where the body movement determination unit 62 determines that the body movement is present in the subject, it is possible to prevent the ultrasound probe 1 from being scanned to the incorrect second position and the second posture due to the body movement of the subject.

It should be noted that the ultrasound diagnostic apparatus according to Embodiment 3 has a configuration in which the body movement determination unit 62 is added to the apparatus body 2 in Embodiment 1, but can also have a configuration in which the body movement determination unit 62 is added to the apparatus body 2A in Embodiment 2.

### Embodiment 4

In Embodiment 3, the body movement determination unit 62 determines the presence or absence of the body movement of the subject based on the plurality of ultrasound images acquired by the image acquisition unit 32, but for example, the presence or absence of the body movement of the subject can also be determined based on an optical image of the subject captured by the optical camera.

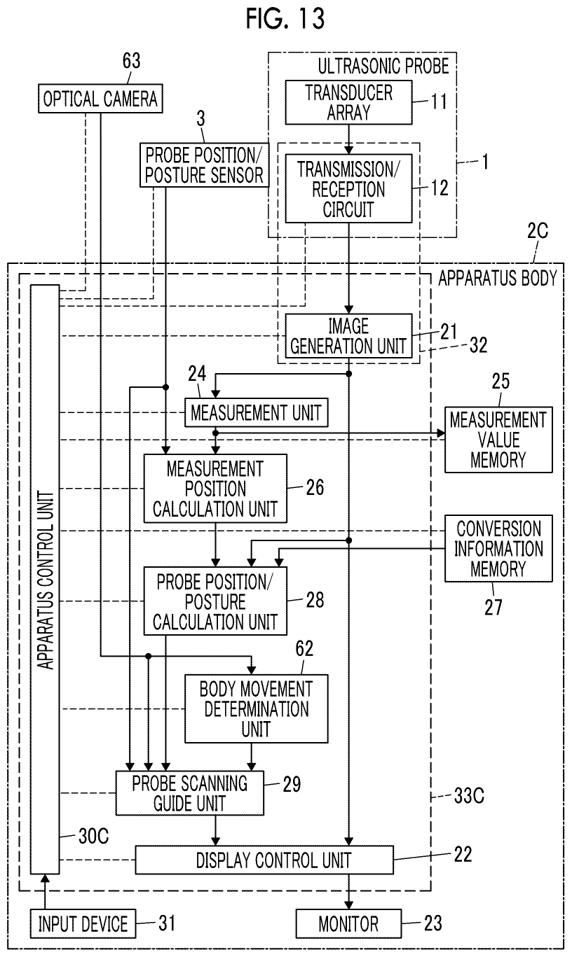

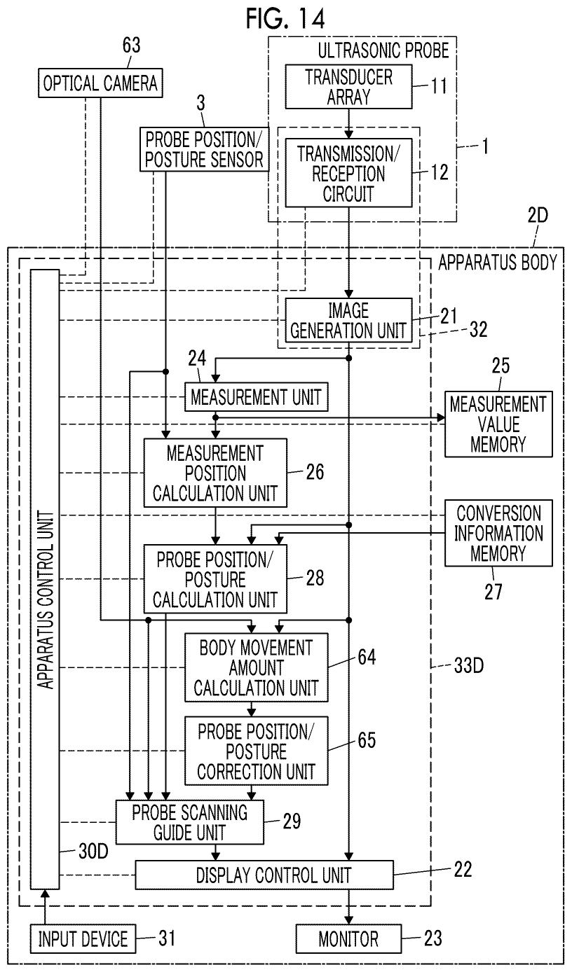

FIG. 13 shows a configuration of an ultrasound diagnostic apparatus according to Embodiment 4. The ultrasound diagnostic apparatus of Embodiment 4 further comprises an optical camera 63 and comprises an apparatus body 2C instead of the apparatus body 2B, with respect to the ultrasound diagnostic apparatus of Embodiment 3 shown in FIG. 12. The apparatus body 2C comprises an apparatus control unit 30C instead of the apparatus control unit 30B in the apparatus body 2B according to Embodiment 3.

In the ultrasound diagnostic apparatus according to Embodiment 4, the optical camera 63 is connected to the apparatus control unit 30. The body movement determination unit 62 and the probe scanning guide unit 29 are connected to the optical camera 63. The body movement determination unit 62 is connected to the probe scanning guide unit 29 and the apparatus control unit 30C. A processor 33C for the apparatus body 2C is configured by the image generation unit 21, the display control unit 22, the measurement unit 24, the measurement position calculation unit 26, the probe position/posture calculation unit 28, the probe scanning guide unit 29, the apparatus control unit 30C, and the body movement determination unit 62.

The optical camera 63 acquires the optical image in which the subject is imaged under the control of the apparatus control unit 30C. The optical camera 63 includes, for example, an image sensor such as a so-called charge coupled device (CCD) image sensor or a so-called a complementary metal-oxide-semiconductor (CMOS) image sensor. The optical camera 63 can be disposed at a fixed position for imaging the subject, and can also be attached to a part of the body of the user such as the head. In addition, in a case where the apparatus body 2C is a handheld device such as a smartphone or a tablet computer, the optical camera 63 can also be attached to the apparatus body 2C.

The body movement determination unit 62 determines presence or absence of the body movement of the subject based on the optical image acquired by the optical camera 63. The body movement determination unit 62 can recognize the subject shown in the optical image by, for example, a method using a trained model in machine learning, calculate a similarity between the subject shown in the current optical image and the subject shown in the past optical image by a certain amount of time before or a certain amount of time before the current optical image by, for example, pattern matching, determine that the body movement is present in a case where the calculated similarity is equal to or less than a certain value, and determine that the body movement is not present in a case where the similarity is greater than the certain value.

The probe scanning guide unit 29 stops the guiding of the scanning with the ultrasound probe 1 in a case where the body movement determination unit 62 determines that the body movement is present. As a result, it is possible to prevent the ultrasound probe 1 from being scanned to the incorrect second position and the second posture due to the body movement of the subject.

As described above, even in a case of determining the presence or absence of the body movement based on the optical image, it is possible to prevent the ultrasound probe 1 from being scanned to the incorrect second position and the second posture due to the body movement of the subject, as in the ultrasound diagnostic apparatus according to Embodiment 3.

It should be noted that the probe scanning guide unit 29 can instruct the user on a scanning direction of the ultrasound probe 1 with the subject as a reference, based on the optical image acquired by the optical camera 63. More specifically, the probe scanning guide unit 29 can acquire, for example, an anatomical positional relationship between each part of the subject, such as a head and a leg of the subject, by analyzing the optical image acquired by the optical camera 63, and can guide the scanning with the ultrasound probe 1 by, for example, “please move the probe by ○ cm to the head side of the patient”, using the acquired anatomical positional relationship. As a result, the user can more accurately understand the direction and the distance in which the ultrasound probe 1 is to be moved. It should be noted that the guiding of the scanning with the ultrasound probe 1 based on the anatomical positional relationship between the parts of the subject can also be performed, for example, by providing the optical camera 63 connected to the probe scanning guide unit 29 in the ultrasound diagnostic apparatus according to Embodiment 1 and the ultrasound diagnostic apparatus according to Embodiment 2.

### Embodiment 5

Embodiments 3 and 4, the guiding of the scanning with the ultrasound probe 1 is stopped in a case where the body movement of the subject occurs, but the second position and the second posture can also be corrected based on the body movement amount of the subject.

FIG. 14 shows a configuration of an ultrasound diagnostic apparatus according to Embodiment 5. The ultrasound diagnostic apparatus according to Embodiment 5 further comprises the optical camera 63 and comprises an apparatus body 2D instead of the apparatus body 2 in the ultrasound diagnostic apparatus according to Embodiment 1 shown in FIG. 1. The optical camera 63 is the same as the optical camera 63 in Embodiment 4. The apparatus body 2D according to Embodiment 5 is different from the apparatus body 2 according to Embodiment 1 in that a body movement amount calculation unit 64 and a probe position/posture correction unit 65 are further provided, and an apparatus control unit 30D is provided instead of the apparatus control unit 30.

In the ultrasound diagnostic apparatus according to Embodiment 5, the body movement amount calculation unit 64 is connected to the image generation unit 21 and the optical camera 63. The probe position/posture correction unit 65 and the apparatus control unit 30D are connected to the body movement amount calculation unit 64. The probe position/posture correction unit 65 is connected to the probe scanning guide unit 29 and the apparatus control unit 30D. In addition, a processor 33D for the apparatus body 2D is configured by the image generation unit 21, the display control unit 22, the measurement unit 24, the measurement position calculation unit 26, the probe position/posture calculation unit 28, the probe scanning guide unit 29, the apparatus control unit 30D, the body movement amount calculation unit 64, and the probe position/posture correction unit 65.

The body movement amount calculation unit 64 can calculate the body movement amount of the subject based on the plurality of ultrasound images acquired by the image acquisition unit 32. The body movement amount calculation unit 64 can calculate, for example, a difference value between the position and the posture of the ultrasound probe 1 in a case of acquiring the past ultrasound image and the position and the posture of the ultrasound probe 1 in a case of acquiring the current ultrasound image as the body movement amount in a case where a similarity between the past ultrasound image after the first ultrasound image is acquired and the current ultrasound image is calculated by pattern matching or the like and the similarity between the past ultrasound image acquired at different positions of the ultrasound probe 1 and the current ultrasound image is greater than a similarity threshold value.

The body movement amount calculation unit 64 can also generate three-dimensional data of the heart based on the plurality of ultrasound images in which the heart of the subject is acquired, and calculate a similarity between a cross section represented by the current ultrasound image and a cross section obtained from three-dimensional data different from the cross section represented by the current ultrasound image by pattern matching or the like. In this case, the body movement amount calculation unit 64 can calculate a difference value between the position and the posture of the ultrasound probe 1 corresponding to a cross section having a similarity greater than a similarity threshold value with respect to the position and the posture of the ultrasound probe 1 in a case of acquiring the current ultrasound image and the cross section represented by the current ultrasound image as the body movement amount.

The body movement amount calculation unit 64 can also calculate the body movement amount of the subject based on the optical image of the subject acquired by the optical camera 63. The body movement amount calculation unit 64 can specify, for example, a position of a chest or the like of the subject shown in the optical image, and calculate a difference value between the position and the posture of the chest or the like before and after the body movement occurs as the body movement amount of the subject.

The probe position/posture correction unit 65 corrects the second position and the second posture of the ultrasound probe 1 calculated by the probe position/posture calculation unit 28 based on the body movement amount calculated by the body movement amount calculation unit 64. The probe position/posture correction unit 65 can correct the second position and the second posture of the ultrasound probe 1 by, for example, adding the body movement amount calculated by the body movement amount calculation unit 64 to the second position of the ultrasound probe 1 calculated before the body movement occurs, that is, in a case where the body movement amount calculated by the body movement amount calculation unit 64 is equal to or less than a body movement amount threshold value.

The probe scanning guide unit 29 guides the scanning with the ultrasound probe 1 based on the second position and the second posture of the ultrasound probe 1 corrected by the probe position/posture correction unit 65 and the current position and the current posture of the ultrasound probe 1 detected by the probe position/posture sensor 3.

As described above, according to the ultrasound diagnostic apparatus according to Embodiment 5, since the probe position/posture correction unit 65 corrects the second position and the second posture of the ultrasound probe 1 in accordance with the body movement of the subject, the scanning with the ultrasound probe 1 can be accurately guided to the second position and the second posture for acquiring the second ultrasound image even in a case where the body movement of the subject occurs.

It should be noted that the ultrasound diagnostic apparatus according to Embodiment 5 has a configuration in which the optical camera 63 is added to the ultrasound diagnostic apparatus in Embodiment 1 and the body movement amount calculation unit 64 and the probe position/posture correction unit 65 are added to the apparatus body 2, but can also have a configuration in which the optical camera 63 is added to the ultrasound diagnostic apparatus in Embodiment 2 and the body movement amount calculation unit 64 and the probe position/posture correction unit 65 are added to the apparatus body 2A.

### Explanation of References

1: ultrasound probe
2, 2A, 2B, 2C, 2D: apparatus body
3: probe position/posture sensor
11: transducer array
12: transmission/reception circuit
21: image generation unit
22: display control unit
23: monitor
24: measurement unit
25: measurement value memory
26: measurement position calculation unit
27: conversion information memory
28: probe position/posture calculation unit
29: probe scanning guide unit
30, 30A, 30B, 30C, 30D: apparatus control unit
31: input device
32: image acquisition unit
33, 33A, 33B, 33C, 33D: processor
41: pulser
42: amplifying unit
43: AD conversion unit
44: beam former
45: signal processing unit
46: DSC
47: image processing unit
48: quadrature detection unit
49: high-pass filter
50: fast Fourier transformation unit
51: Doppler waveform image generation unit
52: data memory
61: conversion information calculation unit
62: body movement determination unit
63: optical camera
64: body movement amount calculation unit
65: probe position/posture correction unit
C1: first cross section
C2: second cross section
G: Doppler gate
M: message
ML: measurement line
P: measurement position
T: left ventricular outflow tract
U1, U2: ultrasound image

What is claimed is:

1. An ultrasound diagnostic apparatus configured to acquire a first ultrasound image and a second ultrasound image in which a first cross section and a second cross section of a heart of a subject, which are different from each other, are respectively imaged, the ultrasound diagnostic apparatus comprising:
an ultrasound probe;
a sensor configured to detect a position and a posture of the ultrasound probe; and
a processor configured to:
calculate a measurement position of a measurement target object in real space based on a first position and a first posture of the ultrasound probe, which are detected by the probe position/posture sensor in a case where the first ultrasound image is acquired, and a measurement position of the measurement target object in the first ultrasound image;
calculate a second posture of the ultrasound probe in a case where the second ultrasound image is acquired from the first posture by using conversion information for converting the first cross section into the second cross section;
calculate a second position of the ultrasound probe in which a cross section drawn by the ultrasound probe at the second posture passes through the measurement position of the measurement target object in the real space; and
guide a user to scan the ultrasound probe such that the ultrasound probe is at the second position and the second posture based on the second position and the second posture of the ultrasound probe and a current position and a current posture of the ultrasound probe.

2. The ultrasound diagnostic apparatus according to claim 1,
wherein one of the first cross section and the second cross section is a parasternal left ventricular long-axis tomographic plane, and the other is a cardiac apex five-chamber tomographic plane.

3. The ultrasound diagnostic apparatus according to claim 1, further comprising:
a memory configured to store predetermined conversion information,
wherein the processor is configured to calculate the second position by using the conversion information stored in the memory.

4. The ultrasound diagnostic apparatus according to claim 1,
wherein the processor is configured to:
calculate the conversion information based on a plurality of ultrasound images in which the heart is imaged; and
calculate the second position and the second posture by using the conversion information.

5. The ultrasound diagnostic apparatus according to claim 4,
wherein the processor is configured to:
generate three-dimensional data of the heart based on the plurality of ultrasound images; and
calculate the conversion information based on the generated three-dimensional data.

6. The ultrasound diagnostic apparatus according to claim 4,
wherein the processor is configured to:
calculate, by performing image analysis, an indicator indicating a second cross section likeness for the plurality of ultrasound images passing through the measurement position of the measurement target object in the real space; and
calculate the conversion information based on the ultrasound image having a highest calculated indicator and the first ultrasound image.

7. The ultrasound diagnostic apparatus according to claim 1,
wherein the processor is configured to:
determine presence or absence of body movement of the subject;
stop the guiding of the scanning with the ultrasound probe upon determining that the body movement is present.

8. The ultrasound diagnostic apparatus according to claim 2,
wherein the processor is configured to:
determine presence or absence of body movement of the subject;
stop the guiding of the scanning with the ultrasound probe upon determining that the body movement is present.

9. The ultrasound diagnostic apparatus according to claim 3,
wherein the processor is configured to:
determine presence or absence of body movement of the subject;
stop the guiding of the scanning with the ultrasound probe upon determining that the body movement is present.

10. The ultrasound diagnostic apparatus according to claim 4,
wherein the processor is configured to:
determine presence or absence of body movement of the subject;
stop the guiding of the scanning with the ultrasound probe upon determining that the body movement is present.

11. The ultrasound diagnostic apparatus according to claim 5,
wherein the processor is configured to:
determine presence or absence of body movement of the subject;
stop the guiding of the scanning with the ultrasound probe upon determining that the body movement is present.

12. The ultrasound diagnostic apparatus according to claim 6,
wherein the processor is configured to:
determine presence or absence of body movement of the subject;
stop the guiding of the scanning with the ultrasound probe upon determining that the body movement is present.

13. The ultrasound diagnostic apparatus according to claim 7,
wherein the processor is configured to determine that the body movement is present upon determining that a similarity between a past ultrasound image and a current ultrasound image is equal to or less than a predetermined similarity threshold value, the position and the posture of the ultrasound probe being the same in the past ultrasound image and the current ultrasound image.

14. The ultrasound diagnostic apparatus according to claim 7,
wherein the processor is configured to:
generate three-dimensional data of the heart based on a plurality of ultrasound images in which the heart is imaged; and
determine that the body movement is present upon determining that a similarity between the current ultrasound image and a two-dimensional image obtained from the three-dimensional data based on a cross section corresponding to the current position and the current posture of the ultrasound probe is equal to or less than a predetermined similarity threshold value.

15. The ultrasound diagnostic apparatus according to claim 7, further comprising:
an optical camera that images the subject,
wherein the processor is configured to determine presence or absence of the body movement of the subject based on an optical image acquired by the optical camera.

16. The ultrasound diagnostic apparatus according to claim 1,
wherein the processor is configured to:
calculate a body movement amount of the subject;
correct the second position and the second posture of the ultrasound probe based on the body movement amount; and
guide the scanning with the ultrasound probe based on the second position and the second posture of the ultrasound probe and the current position and the current posture of the ultrasound probe.

17. The ultrasound diagnostic apparatus according to claim 2,
wherein the processor is configured to:
calculate a body movement amount of the subject;
correct the second position and the second posture of the ultrasound probe based on the body movement amount; and
guide the scanning with the ultrasound probe based on the second position and the second posture of the ultrasound probe and the current position and the current posture of the ultrasound probe.

18. The ultrasound diagnostic apparatus according to claim 3,
wherein the processor is configured to:
calculate a body movement amount of the subject;
correct the second position and the second posture of the ultrasound probe based on the body movement amount; and
guide the scanning with the ultrasound probe based on the second position and the second posture of the ultrasound probe and the current position and the current posture of the ultrasound probe.

19. The ultrasound diagnostic apparatus according to claim 1, further comprising:
an optical camera configured to image the subject,
wherein the processor is configured to instruct the user on a scanning direction of the ultrasound probe with the subject as a reference, based on an optical image acquired by the optical camera.

20. A control method of an ultrasound diagnostic apparatus that acquires a first ultrasound image and a second ultrasound image in which a first cross section and a second cross section of a heart of a subject, which are different from each other, are respectively imaged, the control method comprising:
detecting a position and a posture of an ultrasound probe;
calculating a measurement position of a measurement target object in real space based on a first position and a first posture of the ultrasound probe, which are detected in a case where the first ultrasound image is acquired, and a measurement position of the measurement target object in the first ultrasound image;
calculating a second posture of the ultrasound probe in a case where the second ultrasound image is acquired from the first posture by using conversion information for converting the first cross section into the second cross section;
calculating a second position of the ultrasound probe in which a cross section drawn by the ultrasound probe at the second posture passes through the measurement position of the measurement target object in the real space; and
guiding a user to perform scanning with the ultrasound probe such that the ultrasound probe is at the second position and the second posture based on the second position and the second posture of the ultrasound probe, which are calculated, and a current position and a current posture of the ultrasound probe, which are detected.
